(19) United States
(12) Patent Application Publication
(10) Pub. No.: US 2009/0053171 A1
(43) Pub. Date: Feb. 26, 2009

(54) Treatment of cellular proliferative disorders

(75) Inventors: Lih-Hwa Hwang, Taipei (TW); Kai-Wen Huang, Taipei (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: National Taiwan University

(21) Appl. No.: 12/229,389

(22) Filed: Aug. 22, 2008

Related U.S. Application Data
(60) Provisional application No. 60/957,865, filed on Aug. 24, 2007.

(51) Int. Cl. A61K 38/20; A61K 38/00; A61K 35/76; A61P 31/00; A61K 31/7088

(52) U.S. Cl. 424/85.2; 514/12; 514/44; 424/93.2

(57) ABSTRACT

Disclosed are composition and methods for treating cellular proliferative disorders.

### CROSS REFERENCES OF RELATED APPLICATIONS

This application claims benefit under 35 USC 119(e) of U.S. provisional application No. 60/957,865 filed on Aug. 24, 2007 entitled “Treatment Of Cellular Proliferative Disorders”, the content of which is incorporated herein by reference in its entirety.

### BACKGROUND

There is a need for agents and methods for treating cellular proliferative disorders, such as cancer. Cancer is the second leading cause of death in the United States, exceeded only by heart disease. Despite recent advances in cancer diagnosis and treatment, surgery and radiotherapy may be curative if a cancer is found early. Otherwise, treatment options are limited. For example, hepatocellular carcinoma (HCC), is always identified clinically at an advanced stage while liver function is already impaired. Surgical resection has been considered the only curable approach, but only a small portion of patients are operative candidates. Most of those who can not tolerate operation receive loco-regional therapy, such as percutaneous ethanol injection and transcatheter arterial chemoembolization. However, a reduced hepatic reservoir resulting from underlying liver cirrhosis or repeated antitumor treatment restricts the use of these therapeutic modalities for HCC (Befeler AS and Di Bisceglie AM. Gastroenterology 2002; 122:1609-1619). Since conventional chemotherapy or radiotherapy is ineffective for treating HCC, no optimal treatment is available for patients who suffered from multiple tumors, distant metastasis, or cancer recurrence after initial treatment.

### SUMMARY

The present invention is based on the unexpected finding that a combination of several factors synergistically reduces the tumor size of HCC.

Accordingly, one aspect of the invention is a method for treating a cellular proliferative disorder in a subject. The method includes administering to a subject in need thereof an effective amount of a first polypeptide or a first nucleic acid encoding the first polypeptide, and an effective amount of a second polypeptide (i.e., a polypeptide different from the first polypeptide) or a second nucleic acid encoding the second polypeptide.

The first polypeptide and the second polypeptide are selected from the group consisting of granulocyte-macrophage colony-stimulating factor (GM-CSF), interleukin-12 (IL-12), endostatin (ED), and pigment epithelium-derived factor (PEDF). In one example, the first and the second polypeptides are GM-CSF and IL-12. The first nucleic acid and the second nucleic acid are preferably expression vectors, e.g., adenoviral vectors, which allow the expression of the first and second polypeptide in a host cell. The cellular proliferative disorder can be non-cancer disorders or cancer, such as liver cancer. Each of the above-mentioned polypeptides or nucleic acids can be administered to a tissue or organ, such as a liver, having the cancer. In one embodiment, the method further includes administering to the subject a third polypeptide, such as ED or a third nucleic acid encoding the third polypeptide. In another embodiment, the method includes administering to the subject a forth polypeptide, PEDF, or a forth nucleic acid encoding the forth polypeptide.

Another aspect of this invention features a pharmaceutical composition including at least two of the above-mentioned polypeptides and a pharmaceutically acceptable carrier. The polypeptides are selected from the group consisting of GM-CSF, IL-12, ED, and PEDF. Also within the scope of this invention is a pharmaceutical composition including at least two of the above-mentioned nucleic acids and a pharmaceutically acceptable carrier. Preferably, the nucleic acids are adenoviral vectors.

The details of one or more embodiments of the invention are set forth in the accompanying drawings and description below. Other advantages, features, and objects of the invention will be apparent from the detailed description and the claims.

### DESCRIPTION OF DRAWINGS

The patent or application file contains at least one drawing executed in color. Copies of this patent or patent application publication with color drawing(s) will be provided by the Office upon request and payment of the necessary fee. FIGS. 1A-1C are diagrams showing synergistic antitumor effects induced by combined IL-12 and GM-CSF gene therapy. Orthotopic liver tumors, implanted or multifocal, were generated and treated with adenoviruses as described in Materials and Methods. Tumor-bearing BALB/c mice were treated on day 7 (A) or day 14 (B) after tumor implantation. Liver tumor sizes were measured on day 28 using calipers. Each group consists of five mice. (C). Wistar rats were fed with DEN for 10 weeks to induce multifocal liver tumors and then treated with adenoviruses. Tumor burdens were expressed as a modified tumor burden index (MTBI), which indicates the difference of the ratio of liver weight/body weight between tumor-bearing rats and normal healthy rats. Each group consists of 10 animals. The reduction fold of tumor volumes or tumor burdens of each treatment compared to that of Ad/GFP treatment is shown at the bottom of each bar. Statistical significance was set at *, p<0.05; **, p<0.005; ***, p<0.001 by Wilcoxon test.

FIG. 2 is a diagram showing serum IFN-γ levels after adenovirus injection. BALB/c mice bearing 7-day-old tumors were treated with adenoviruses as described in Materials and Methods. Serum IFN-γ levels were determined by ELISA after adenoviruses or PBS injection at the time indicated. Each group consists of three mice. On day 6, the IFN-γ levels of Ad/combined group were significantly higher than that of Ad/IL-12 group (p=0.00105, one-way ANOVA).

FIGS. 3A-3C are diagrams showing roles of CD4+, CD8+, NKT, and NK cell subsets in the IL-12- or combination therapy-mediated antitumor effects. (A). Cell subset depletion. BALB/c mice bearing 7-day-old tumors were i.p. injected with anti-CD4, anti-CD8, or anti-asialoGM1 antibody according to the protocols described in Materials and Methods. Splenocytes were isolated on day 20 after tumor implantation, one day after the last antibody injection, and depletion efficiency of each cell subset was determined by flow cytometry. Each group consists of three mice. Tumor growth under specific cell subset depletion was re-examined in Ad/IL-12-treated (B) and Ad/combined-treated animals (C), as described in FIG. 1. Tumor sizes were measured on day 28 after tumor implantation. ‘PBS’ means the tumors were not treated with adenovirus. ‘None’ means the tumors were treated with respective adenoviruses but without cell subset depletion. Other bars represent the tumor sizes of the animals treated with Ad/IL-12 or Ad/combined and depleted of CD4, CD8 T cells, or NK cells, respectively. IgG2a is a mouse mAb control and rabbit serum is a normal rabbit serum control. Each group consists of five mice. Significant tumor regrowth compared to IgG2a or normal rabbit serum control is indicated by stars; *, p<0.05; **, p<0.005; ***, p<0.001.

FIGS. 4A-4C. Diverse effectors induced by Ad/IL-12 or Ad/combined treatment. Mononuclear cells were isolated from the tumors of the animals treated with adenoviruses or PBS on day 4 after adenovirus injection. (A). IFN-γ-secreting effector cells. Cells were stained with antibody against CD4, CD8, or NK cells or with α-GalCer-loaded CD1d DimerX I, followed by intracellular IFN-γ staining. (B). CD1d-expressing DCs. Cells were doubly stained with anti-CD1d and anti-CD11c antibodies. (C). Tumor specific CD8+ T cells. TILs isolated were in vitro stimulated with irradiated BNL cells (black bars) or without BNL cells (white bars) for 24 h, followed by intracellular IFN-γ staining. IFN-γ+ cells were counted by flow cytometry. (D). Cytolytic NK activity. Splenocytes were isolated on day 4 after adenovirus injection from the animals treated with adenoviruses or PBS, and assayed against YAC-1 cells. Cell lysis was determined in triplicate by LDH assay at different effector/target ratios. The bars represent mean cell number±SD/mg tumor tissue of the double positive cells. Each group consists of five mice. The figures show one representative data of two independent experiments. *, p<0.05; **, p<0.005; ***, p<0.001, compared to Ad/GFP (one-way ANOVA).

FIGS. 5A and 5B are pictures showing NKT cells in tumor infiltrating lymphocytes. (A). High proportion of CD4+ cells in the TILs are invariant NKT cells. TILs were triply stained with anti-IFN-γ, anti-CD4, and α-GalCer-loaded CD1d DimerX I. CD4+IFN-γ+ cells were gated and further analyzed for NKT T cell receptor expression by CD1d DimerX I staining. (B). Significant activation of CD4/CD8 double negative NKT cells by Ad/combined treatment. TILs were stained as described in (A). NKT+IFN-γ+ cells were gated and further analyzed for CD4 expression by anti-CD4 staining. Isotype-matched antibodies were used as negative controls in flow cytometry analysis. Quadrants were set according to baseline signal given by control antibodies.

FIGS. 6A and 6B are photographs and diagram showing macrophages and iNOS expression at the tumor sites of the animals treated with adenoviruses or PBS. (A) Macrophage infiltration in the tumor regions after adenovirus treatment. Four days after adenoviruses or PBS treatment, mice were killed and the tumor sites were sectioned and stained for macrophages and iNOS using anti-Mac-3 and anti-iNOS, respectively. (B). Reduced tumor infiltrating macrophages in the mice depleted of IFN-γ. Mice were i.p. injected with anti-IFN-γ or control IgG2a before, at, and after Ad/combined injection. On day 4, tumor infiltrating cells were isolated and analyzed by surface staining with anti-CD11b antibody, followed by intracellular staining with anti-iNOS antibody. The bars represent mean cell number±SD/mg tumor tissue of the double positive cells. Each group consists of 4 mice. ***, p<0.001, anti-IFN-γ vs. IgG2a depletion control (ANOVA).

FIGS. 7A and 7B are diagrams showing synergistic antitumor effects induced by combined ED and PEDF gene therapy. Orthotopic liver tumors, implanted or multifocal, were generated and treated with adenoviruses as described in Materials and Methods. (A) Tumor-bearing BALB/c mice were treated on day 7 after tumor implantation. Liver tumor sizes were measured on day 28 using calipers. Each group consists of five mice. (B). Wistar rats bearing primary multifocal liver tumors were treated with adenoviruses. Tumor volumes were measured two weeks after adenovirus treatment. The magnification of tumor burden scales is shown in the inset. Each group consists of 5 animals. The reduction fold of tumor volumes or tumor burdens of each treatment compared to that of Ad/GFP treatment is shown at the bottom of each bar. Statistical significance was set at *, p<0.05; **, p<0.005; ***, p<0.001 by Wilcoxon test.

FIGS. 8A and 8B are diagrams showing synergistic antitumor effects induced by the 4-in-1 therapy. (A) Tumor-bearing BALB/c mice were treated on day 14 after tumor implantation, representing the large liver tumors. Liver tumor sizes were measured on day 28 using calipers. Each group consists of five mice. (B) The multifocal tumor model was used to further verify the synergistic anti-tumor effect of the 4-in-1 therapy. Tumor volumes were measured two weeks after adenovirus treatment. The magnification of tumor burden scales is shown in the inset. Each group consists of 5 animals. The reduction fold of tumor volumes or tumor burdens of each treatment compared to that of Ad/GFP treatment is shown at the bottom of each bar. Statistical significance was set at *, p<0.05; **, p<0.005; ***, p<0.001 by Wilcoxon test.

FIGS. 9A-9D show the immunohistochemical staining of tumor regions. (A) The 4-in-1 therapy reduced microvessel density (MVD) in tumor regions. The MVD was quantified by counting the vessel number from 10 random fields. (B) The 4-in-1 therapy induced high apoptosis in tumor region. TUNEL-positive cells were counted from 10 random fields to determine the apoptotic index. (C) The 4-in-1 therapy also induced significantly higher numbers of tumor infiltrating CD4-positive T cells and (D) CD8-positive T cells to the tumor regions. Statistically significance was set as *, p<0.05, **, p<0.005; ***, p<0.001. compared with GFP group by one-way ANOVA.

### DETAILED DESCRIPTION

Described below are methods of this invention for treating a cellular proliferative disorder, such as cancer, in a subject. The methods use at least two of GM-CSF, IL-12, ED, and PEDF.

GM-CSF is a cytokine that functions as a white blood cell growth factor. It stimulates stem cells to produce granulocytes (neutrophils, eosinophils, and basophils) and monocytes. IL-12 is an interleukin, or hormone of the immune system that is instrumental in the body's natural response to microbial infection and in discriminating between foreign and self. Endostatin is a naturally-occurring 20-kDa C-terminal fragment derived from type XVIII collagen. It is reported to serve as an anti-angiogenic agent, similar to angiostatin and thrombospondin. It is a broad spectrum angiogenesis inhibitor and may interfere with the pro-angiogenic action of growth factors such as basic fibroblast growth factor (bFGF/FGF-2) and vascular endothelial growth factor (VEGF). Pigment epithelium-derived factor is also a potent inhibitor of angiogenesis. It targets tumors through various destructive pathways, including anti-proliferation, anti-angiogenesis, and pro-differentiation.

While many preparations of the above-mentioned GM-CSF, IL-12, ED, or PEDF can be used, highly purified GM-CSF, IL-12, ED, or PEDF is preferred. Examples of these polypeptides include mammalian polypeptides (e.g., those of human) or polypeptides having substantially the same biological activity as mammalian GM-CSF, IL-12, ED, or PEDF. All of naturally occurring, genetic engineered, and chemically synthesized GM-CSF, IL-12, ED, or PEDF. Polypeptides obtained by recombinant DNA technology may have the same amino acid sequence as naturally occurring GM-CSF, IL-12, ED, or PEDF or an functionally equivalent thereof. The term “GM-CSF, IL-12, ED, or PEDF” also covers chemically modified GM-CSF, IL-12, ED, or PEDF. Examples include GM-CSF, IL-12, ED, or PEDF subjected to conformational change, addition or deletion of a sugar chain, and to which a compound such as polyethylene glycol has been bound. Once purified and tested by standard methods or according to the method described in the examples below, GM-CSF, IL-12, ED, or PEDF can be included in a composition.

The amino acid composition of the GM-CSF, IL-12, ED, or PEDF polypeptide described herein may vary without disrupting the ability of the polypeptide. For example, it can contain one or more conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in the sequence of GM-CSF, IL-12, ED, or PEDF is preferably replaced with another amino acid residue from the same side chain family. Alternatively, mutations can be introduced randomly along all or part of the sequence, such as by saturation mutagenesis, and the resultant mutants can be screened for the activities of GM-CSF, IL-12, Endostatin, or PEDF. The methods for testing these activities are known in the art.

The aforementioned polypeptides can be synthesized using methods known in the art or be prepared using recombinant technology. For example, one can clone a nucleic acid encoding the polypeptide in an expression vector, in which the nucleic acid is operably linked to a regulatory sequence suitable for expressing the polypeptide in a host cell. One can then introduce the vector into a suitable host cell to express the polypeptide. The expressed recombinant polypeptide can be purified from the host cell by methods such as ammonium sulfate precipitation and fractionation column chromatography. A polypeptide thus prepared can be tested for its activity according to the method described in the examples below.

The invention features methods for treating a cellular proliferative disorder (e.g., cancer) in a subject. A cellular proliferative disorder refers to a disorder characterized by uncontrolled, autonomous cell growth, including malignant and non-malignant growth. Examples of this disorder include liver cancer (e.g., HCC), colon cancer, breast cancer, prostate cancer, hepatocellular carcinoma, melanoma, lung cancer, glioblastoma, brain tumor, hematopoeitic malignancies, retinoblastoma, renal cell carcinoma, head and neck cancer, cervical cancer, pancreatic cancer, esophageal cancer, and squama cell carcinoma.

A subject refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

A subject to be treated for a cellular proliferative disorder can be identified by standard diagnosing techniques for the disorder. “Treating” refers to administration of a compound or composition to a subject, who has a cellular proliferative disorder (e.g., cancer) with the purpose to cure, alleviate, relieve, remedy, prevent, or ameliorate the disorder, the symptom of the disorder, the disease state secondary to the disorder, or the predisposition toward the disorder. An “effective amount” refers to an amount of the compound that is capable of producing a medically desirable result, e.g., as described above, in a treated subject. The treatment method can be performed in vivo or ex vivo, alone or in conjunction with other drugs or therapy.

In an in vivo approach, a compound or composition is administered to a subject. Generally, the compound is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The above-mentioned nucleic acids or polynucleotide can be delivered by the use of polymeric, biodegradable microparticle or microcapsule delivery devices known in the art. Another way to achieve uptake of the nucleic acid is using liposomes, prepared by standard methods. The polynucleotide can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al., 1995, J. Mol. Med. 73:479). Alternatively, tissue specific targeting can be achieved by the use of tissue-specific transcriptional regulatory elements that are known in the art. Delivery of “naked DNA” (i.e., without a delivery vehicle) to an intramuscular, intradermal, or subcutaneous site is another means to achieve in vivo expression.

In the above-mentioned polynucleotides, e.g., expression vectors, the nucleic acid sequence encoding GM-CSF, IL-12, ED, or PEDF is operatively linked to a promoter or enhancer-promoter combination. Suitable expression vectors include plasmids and viral vectors such as herpes viruses, retroviruses, vaccinia viruses, attenuated vaccinia viruses, canary pox viruses, adenoviruses and adeno-associated viruses.

As is well known in the art, the dosage for a patient depends upon various factors as described above. Dosages will vary, but a preferred dosage for administration of polynucleotide is about 10⁶ to 10¹² copies of the polynucleotide molecule. This dose can be repeatedly administered as needed. Routes of administration can be any of those listed above.

Within the scope of this invention is a composition that contains a suitable carrier and one or more of the compounds described above. The composition can be a pharmaceutical composition that contains a pharmaceutically acceptable carrier or a cosmetic composition that contains a cosmetically acceptable carrier.

A composition of the present invention may include a carrier. Depending on the kind of the composition, a carrier may be a pharmaceutically acceptable carrier. Examples of a pharmaceutically acceptable carrier include, but are not limited to, biocompatible vehicles, adjuvants, additives, and diluents to achieve a composition usable as a dosage form. A pharmaceutically acceptable carrier, after administered to or upon a subject, does not cause undesirable physiological effects. The carrier in the pharmaceutical composition must be acceptable also in the sense that it is compatible with the active ingredient and, preferably, capable of stabilizing it. One or more solubilizing agents can be utilized as pharmaceutical carriers for delivery of an active compound. The above-described composition, in any of the forms described above, can be used for treating cellular proliferative disorders.

An “effective amount” refers to the amount of an active compound that is required to confer a therapeutic effect on a treated subject. Effective doses will vary, as recognized by those skilled in the art, depending on the types of diseases treated, route of administration, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having an active compound can also be administered in the form of suppositories for rectal administration.

A topical composition contains a safe and effective amount of a dermatologically acceptable carrier suitable for application to the skin. Generally, a topical composition can be solid, semi-solid, cream, or liquid. It may be a cosmetic or dermatologic product in the form of an ointment, lotion, foam, cream, gel, or solution. Details about dermatologically acceptable carriers are provided below.

A composition of the present invention may be used alone or in combination with other biologically active ingredients. Alone or in combination with other active ingredients, it may be administered to a subject in a single dose or multiple doses over a period of time. Various administration patterns will be apparent to those skilled in the art. The dosage ranges for the administration of the composition are those large enough to produce the desired effect. The dosage should not be so large as to cause any adverse side effects, such as unwanted cross-reactions and the like. Generally, the dosage will vary with the age, weight, sex, condition, and extent of a condition in a subject, and the intended purpose. The dosage can be determined by one of skill in the art without undue experimentation. The dosage can be adjusted in the event of any counter indications, tolerance, or similar conditions. Those of skill in the art can readily evaluate such factors and, based on this information, determine the particular effective concentration of a composition of the present invention to be used for an intended purpose.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Immunotherapy has been thought to have significant advantages when applied to cancers, particularly multifocal tumor nodules or tumor metastasis. The successful immunosurveillance makes it an ideal tool for eradicating tumors systematically. Additionally, the established immunological memory following active vaccination with tumor associated antigens or tumor cell vaccines often induces persisting tumor-specific T-cells, providing a system for long-term prevention of cancer recurrence. Cytokines are often used in immunotherapy to augment antitumor immunity. Among them, granulocyte macrophage colony-stimulating factor (GM-CSF) is one of the most potent cytokines in cancer treatment (Dranoff G et al. Proc Natl Acad Sci USA 1993; 90:3539-3543; Hsieh CL et al. Hum Gene Ther 1997; 8:1843-1854). The underlying mechanisms of GM-CSF action mainly involve the enhanced capacity of antigen presentation of dendritic cells induced by GM-CSF, which subsequently activates tumor-specific cytotoxic T lymphocytes (CTL) and causes tumor regression (Tazi A et al. J Clin Invest 1993; 91:566-576). Interleukin (IL)-12 is another potent antitumor cytokine that can augment the cytotoxic activity of CTLs, natural killer (NK) cells or natural killer T (NKT) cells against a wide variety of target cells, and induces them to secrete interferon (IFN)-γ (Brunda M J. J Leukoc Biol 1994; 55:280-288; Nastala CL et al. J Immunol 1994; 153:1697-1706).

Anti-angiogenesis therapy for cancer can effectively inhibit tumor growth by inhibiting tumor-related angiogenesis, and thus deprive tumors of essential nutrients and oxygen, which leads to a ‘dormant’ state in which tumor cell proliferation and metastasis are halted. There are two major improvements of anti-angiogenic therapy over traditional cancer treatments. First, the anti-angiogenesis therapy is mainly inhibits the growth of new capillaries. This advance is more moderate than traditional chemotherapy due to its specificity to endothelial cells and it has no harm to the normal cells. Second, drug resistance becomes the major obstacles in cancer treatment due to their genetic instability and consequent plasticity of their genome. The anti-angiogenesis targeted at normal and stable endothelial cells in which mutations and drug resistance are less likely.

We have previously reported the effectiveness of a recombinant adenovirus carrying a GM-CSF and an endostatin genes on early rat HCC model; however, the efficacy was quite disappointed in the treatment of large tumors (Tai K F et al. J Gene Med 2003; 5:386-398). Similarly, the efficacy of IL-12 single gene therapy, though significant in treating HCC, is also limited to small tumor burdens and, in many cases, restricted to subcutaneous (s.c.) tumor models. To augment the antitumor effects of gene therapy on larger tumor burdens, especially on orthotopic liver tumors, this study performed immunotherapy by adenovirus-mediated gene transfer of IL-12 and GM-CSF simultaneously, anti-angiogenesis therapy by adenovirus-mediated gene transfer of ED and PEDF simultaneously, or combination therapy with all the four adenoviruses together, to animals bearing large hepatic tumors or animals with chemically-induced multiple HCC nodules. Our results show that in situ tumor therapy with co-administration of Ad/GM-CSF and Ad/IL-12, or of Ad/ED and Ad/PEDF, synergistically reduces tumor volumes, compared to single gene therapy. Moreover, combination of immunotherapy and anti-angiogenesis therapy by adenovirus-mediated gene transfer of GM-CSF, IL-12, ED, and PEDF exhibited even better therapeutic effects on tumor regression compared to immunotherapy (GM-CSF+IL-12) or anti-angiogenesis therapy (ED+PEDF) alone at the same viral dose. Our findings suggest that the co-action of two different treatment strategies can more effectively reduce tumor burdens compared to single treatment strategy.

### EXAMPLES

### Materials and Methods

### Cell Lines and Animals

The mouse hepatoma cell line BNL and human embryonic kidney cell line 293 were purchased from the American Type Culture Collection (Rockville, Md.). Both cell lines were maintained in Dulbecco's modified Eagle's medium (DMEM; Seromed, Berlin, Germany) supplemented with 10% fetal calf serum (FCS; Biological Industries, Israel). Male BALB/c mice aged 7-8 weeks and male Wistar rats aged 6-7 weeks were used in these experiments. All animal experiments were performed in accordance with the guidelines of the Animal Welfare Committee of National Taiwan University College of Medicine.

### Construction of Adenoviral Vector

The adenoviral vector containing a mouse GM-CSF cDNA (Ad/GM-CSF) or a GFP gene (Ad/GFP) under the control of a CMV immediate early gene promoter was constructed using the AdEasy system (He TC et al. Proc Natl Acad Sci USA 1998; 95:2509-2514) as described previously (Tai K F et al. J Gene Med 2003; 5:386-398). Ad/IL-12, kindly provide by Dr. B. L. Chiang of National Taiwan University College of Medicine, is the adenoviral vector containing a murine single-chain IL-12 gene which encodes the two IL-12 subunits (p35 and p40) linked by a polypeptide linker (Lee Y L et al. Hum Gene Ther 2001; 12:2065-2079).

### Example 1

### Generation of Orthotopic Liver Tumors and In Vivo Gene Therapy

For single HCC nodule model, 3×10⁵ BNL cells were injected into the left liver lobe of mice on day 0. The needle hole was sealed with an electric coagulator (Aaron, Petersburg, Fla., USA) immediately after the withdrawal of the needle to avoid leakage of the injected substance. The incision was subsequently sutured. A single injection of 30 μl of adenoviruses, 2×10⁹ Ad/GFP, 2×10⁹ Ad/GM-CSF, 2×10⁹ Ad/IL-12, 1×10⁹ Ad/GM-CSF+1×10⁹ Ad/IL-12, or 30 μl PBS was administered intratumorally on day 7 or day 14 (n=5 for each group) after tumor implantation. Tumors were measured using calipers on day 28 by an investigator blinded to the treatment groups. Tumor volume was calculated using the formula: volume=width²×length×0.52.

For primary multifocal HCC model, Wistar rats received 0.02 ml/kg/day of diethyInitrosamine (DEN)(Sigma, St Louis, Mo., USA) for 10 weeks by giving weekly doses of DEN in a volume corresponding to the estimated water consumption of 7 days of drinking water (100 ppm). The weights of the rats were recorded and DEN solution was freshly prepared every week. After 10 weeks, a PE10 silicon tube was inserted into the gastroduodenal artery of the animal using an operating microscope (×20 magnification). A single injection of adenovirus (same dosages as described above) in 100 μl or 100 μl PBS (n=10 for each group) was infused through the hepatic artery into the liver by the silicon tube, then the gastroduodenal artery was ligated. Two weeks after treatment, rats were sacrificed and livers were harvested and weighed. Another 10 age-matched Wistar rats without DEN feeding were sacrificed at the same time and used as healthy control group. Therapeutic effects were determined by the modified tumor burden index which was defined as the difference of the ratios of liver weight/body weight between the treated groups and the healthy controls.

### Antibody-Mediated Depletion of CD4+ or CD8+ T-Cells, NK Cells or IFN-γ

BNL cells (3×10⁵) were inoculated in the liver of BALB/c mice on day 0. Adenoviruses were intratumorally injected on day 7 post tumor implantation. CD4+ and CD8+ T-cells or IFN-γ were depleted by intraperitoneally (i.p.) injection of 0.5 mg of anti-CD4 monoclonal antibody (mAb) (GK1.5), anti-CD8 mAb (53-6.72), or anti-IFN-γ mAb (R4-6A2), respectively, on day 5 (i.e., two days before the administration of adenoviruses), and then with 0.25 mg of the same mAb on days 8, 10, 12, and 19 after tumor implantation. NK cells were depleted by i.p. injection of 20 μl of rabbit anti-asialoGM1 antiserum (Wako, Osaka, Japan) using the same schedule. Mice i.p. injection with normal rat IgG or normal rabbit serum at the same dose and schedule were used as controls. Depletion of CD4+, CD8+, or NK cells was confirmed by flow cytometry. Tumor growth in each group was monitored on day 28 after tumor implantation.

### Flow Cytometric Analysis of Tumor-Infiltrating Lymphocytes (TILs)

TILs from tumor tissues were prepared on day 4 after adenovirus injection as described previously (Chang CJ et al. J Immunol 2004; 173:6025-6032). Briefly, resected liver tumors were cut into small pieces using a razor blade. The tissue fragments were incubated for 15 min at 37° C. in HBSS solution (1 g/10 ml) containing collagenase type I (0.05 mg/ml), collagenase type iV (0.05 mg/ml), hyaluronidase (0.025 mg/ml) and soybean trypsin inhibitor (1 mg/ml) (all from Sigma-Aldrich) and DNase I (0.01 mg/ml; Roche Applied Science). Cells were recovered by centrifugation and suspended again in a fresh aliquot of the HBSS digestion solution for 15 min at 37° C. Undigested material was removed on a 40-μm mesh sieve, and the liberated cells were recovered and washed with RPMI 1640 medium. They were further separated on a Ficoll-Paque gradient to remove dead cells. The cells obtained were used for cytometric analysis. The surface markers of these cells were stained with directly conjugated antibodies (all from BD Biosciences Pharmingen): isothiocyanate (FITC)-conjugated anti-CD4 mAb (GK1.5), anti-CD8 mAb (53-6.72), or phycoerythrin (PE)-conjugated anti-CD3 mAb (145-2C11). NKT cells were detected with α-GalCer-loaded DimerX I (CD1d:Ig fusion protein) (BD Biosciences Pharmingen) which was probed with PE-conjugated A85-1 mAb (anti-mouse IgG). After surface staining, cells were fixed and permeabilized according to the manufacturer's protocol (BD Biosciences Pharmingen), and then stained with Alexa Fluor647-conjugated anti-IFN-γ mAb (XMG1.2) or isotype-matched control Ab. The stained cells were analyzed with a FACScan (Becton Dickinson, Mountain View, Calif.), and the data were processed using CELLQest Software (BD Biosciences Pharmingen).

### In Vitro Activation of Tumor-Specific CD8+ T Cells

To determine BNL-specific CD8+ T cells, 1×10⁵ TILs were activated by incubating with 1×10⁵ irradiated BNL at 37° C. for 24 h in the presence of 20 ng/ml IL-2, 1 μg/ml anti-CD28, and 2 μM monensin. After overnight activation, the cells were stained with anti-CD8 antibody, followed by intracellular IFN-γ staining as described above.

### NK Activity Assay

NK cytolytic activity was determined by lactate dehydrogenase (LDH) assay (Promega, Madison, Wis.) using YAC cells as target cells at the E/T ratios indicated according to the manufacturer's instructions. The percentage of specific lysis was calculated by the following formula: % cytotoxicity=[(experimental LDH release−spontaneous LDH release by effector and target)/(maximal LDH release−spontaneous LDH release)]×100. Target cells were incubated either in culture medium alone to determine spontaneous LDH release or in a mixture of 2% Triton X-100 to define maximal LDH release. All assays were performed in triplicate.

### Statistical Analysis

All results are expressed as means±SE. One-way ANOVA was used to evaluate the statistical significance of the difference in tumor volumes between different groups.

### Results

### Adenoviral Delivery of GM-CSF and IL-12 Synergistically Regresses Orthotopic Liver Tumors

To test immunotherapy strategies in a clinically relevant situation, this study used orthotopic liver tumor models representing either an intermediate or a large tumor load. BNL cells (3×10⁵) were injected in the left liver lobe of BALB/c mice. Usually, a tumor nodule of 10˜20 mm³ and 60˜100 mm³ could be observed on day 7 and day 14, respectively, after tumor implantation, representing the intermediate tumor burden and the large tumor burden, respectively. A single injection of adenoviruses (Ad/GFP, Ad/GM-CSF, Ad/IL-12, or Ad/GM-CSF+Ad/IL-12) was administered intratumorally to the 7-day-old and 14-day-old tumors. Liver tumors were measured on day 28 using calipers. As shown in FIG. 1, while animals treated with Ad/GM-CSF only had marginal effects, those treated with Ad/IL-12 showed significant tumor reduction either in the 7 day-old tumor model (FIG. 1A, p<0.001) or in the 14 day-old tumor model (FIG. 1B, p<0.05), compared to the control PBS or Ad/GFP-treated group. Remarkably, animals treated with Ad/GM-CSF+Ad/IL-12 (i.e., Ad/combined) almost completely regressed tumor in the 7 day-old tumor model (p<0.001) and synergistically reduced tumor volumes in the 14 day-old tumor model (p<0.001) compared to either monotherapy.

### GM-CSF and IL-12 Combination Therapy Significantly Regresses Multifocal HCC Induced by DEN in Rats

We further pursued the antitumor effects of cytokine immunotherapy on a multifocal liver tumor model. Primary liver tumors were induced in Wistar rats with DEN as described previously (Barajas M et al. Hepatology 2001; 33:52-61). Normally, multifocal tumors were generated within a 6˜8 week period. Adenoviruses or PBS were injected via hepatic artery. The liver weights and body weights of the rats were measured two weeks after treatment. Tumor burden was expressed as a modified tumor burden index (MTBI), which indicates the difference of the ratio of liver weight/body weight between tumor-bearing rats and normal healthy rats. Usually, normal healthy animals have a constant liver weight/body weight ratio; whereas animals bearing liver tumors have higher ratios than healthy ones. In our model, the mean ratio of liver weight/body weight of healthy rats was around 0.0405±0.004 (n=10), whereas that of tumor-bearing animals treated with PBS increased to 0.0697±0.01 (n=9). Thus, the MTBI of the PBS control group is 0.0292±0.0123 (FIG. 1C). In contrast, the MTBI of the Ad/combined-treated animals was very close to that of healthy animals, and was synergistically reduced (92% reduction) compared to Ad/GM-CSF (26% reduction) or Ad/IL-12 (55% reduction) monotherapy (FIG. 1C). These results demonstrate that combination therapy with GM-CSF and IL-12 also has enormous effects on the multifocal HCC model.

### GM-CSF and IL-12 Combination Therapy Induces Significantly High Levels of IFN-γ

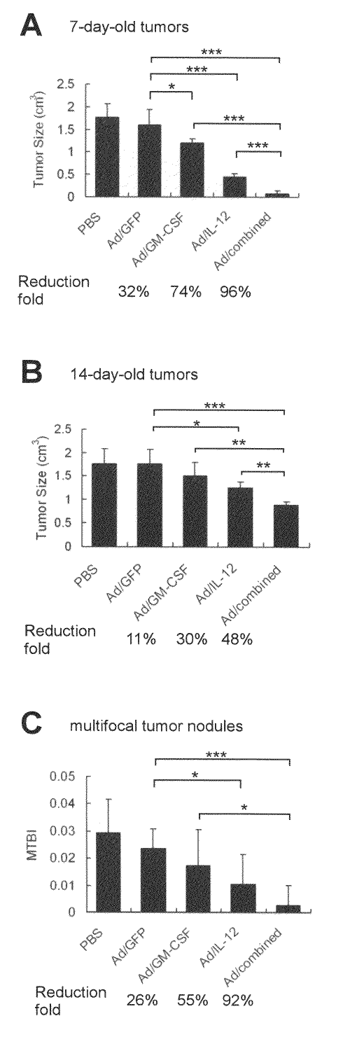

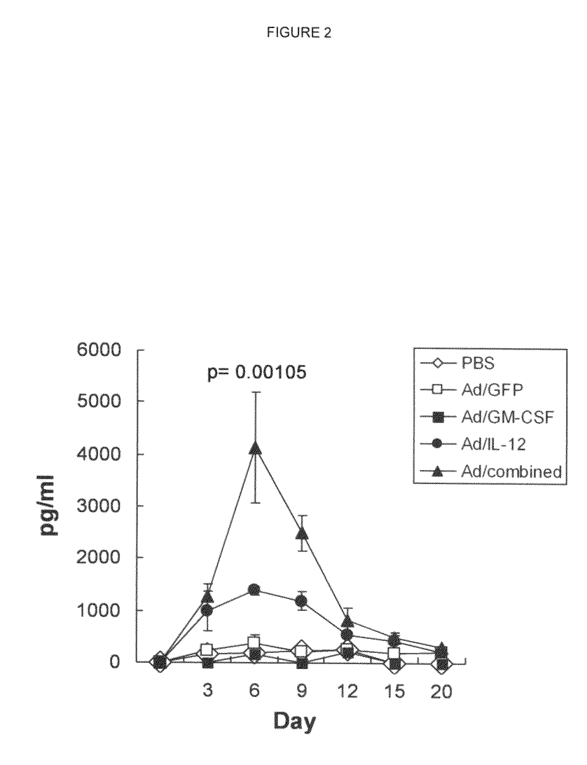

IFN-γ is a main cytokine induced by IL-12 and is critically involved in the development of cell-mediated immune responses, so we analyzed IFN-γ production in the animals treated with adenoviruses. As shown in FIG. 2, the serum levels of IFN-γ in Ad/IL-12-treated BLAB/c mice were high (˜1,000 pg/ml), which lasted for about 12 to 20 days. Notably, the serum levels of IFN-γ in Ad/combined-treated group were about four folds of those of Ad/IL-12-treated group (˜4,000 pg/ml). These results thus demonstrate that combined administration of Ad/GM-CSF and Ad/IL-12 greatly enhances IFN-γ production.

### Ad/IL-12 and Ad/Combined Treatments Employ Different Effector Cells for Tumor Regression

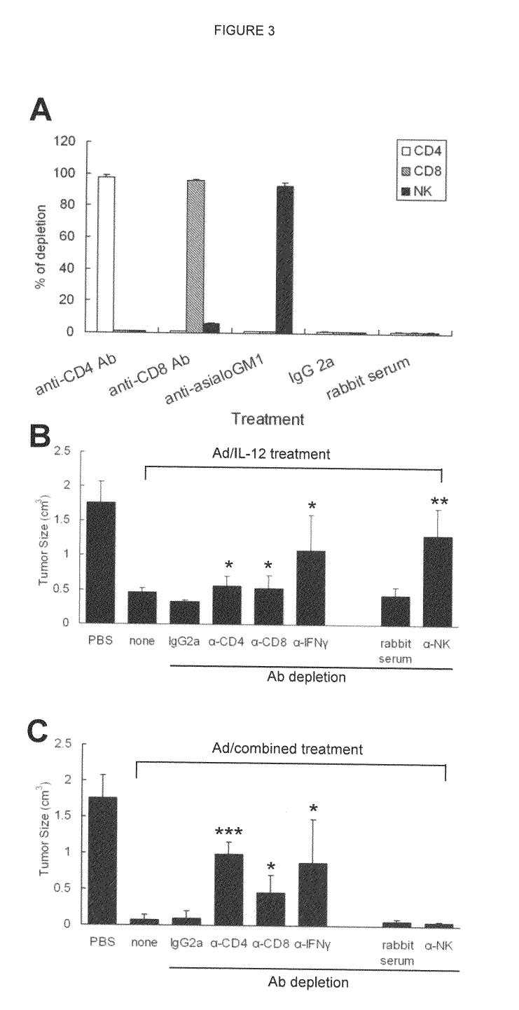

To further assess the effectors involved in IL-12-mediated or combination therapy-mediated antitumor immunity, BALB/c mice were depleted of CD4+ or CD8+ T-cells, or NK cells by anti-CD4 or anti-CD8 mAb or anti-asialoGM1 antiserum, respectively. Mice treated with an irrelevant rat monoclonal IgG2a or normal rabbit serum at the same dose and schedule were included as controls. The depletion efficiency of each cell subset by respective antibody is shown in FIG. 3A, which shows a 97.9%, 96.2%, and 93.1% depletion for CD4+, CD8+ T cells and NK cells, respectively; whereas depletion of IFN-γ was nearly 99% as measured by ELISA (data not shown). In the animals treated with Ad/IL-12 depletion of NK cells significantly impaired the antitumor effects of Ad/IL-12 (FIG. 3B, p<0.005). Depletion of CD4+ or CD8+ T-cells also had some, but minor, effects on antitumor activity (p<0.05). On the contrary, in animals treated with Ad/combined depletion of NK cells had no effect at all on antitumor activity (FIG. 3C), whereas tumor growth was greatly restored in the CD4+ T-cell-depleted mice (p<0.001), and was partially restored in the CD8+ T-cell-depleted mice (p<0.05). In both treatments, neutralization of IFN-γ greatly impaired the antitumor effects (FIGS. 3B & C), establishing that IFN-γ is critical to the antitumor effects of Ad/IL-12 and Ad/combined treatments.

Ad/Combined Treatment Elicits Much Higher Levels of Diverse Effectors in the Tumor Regions than Ad/IL-12 Treatment

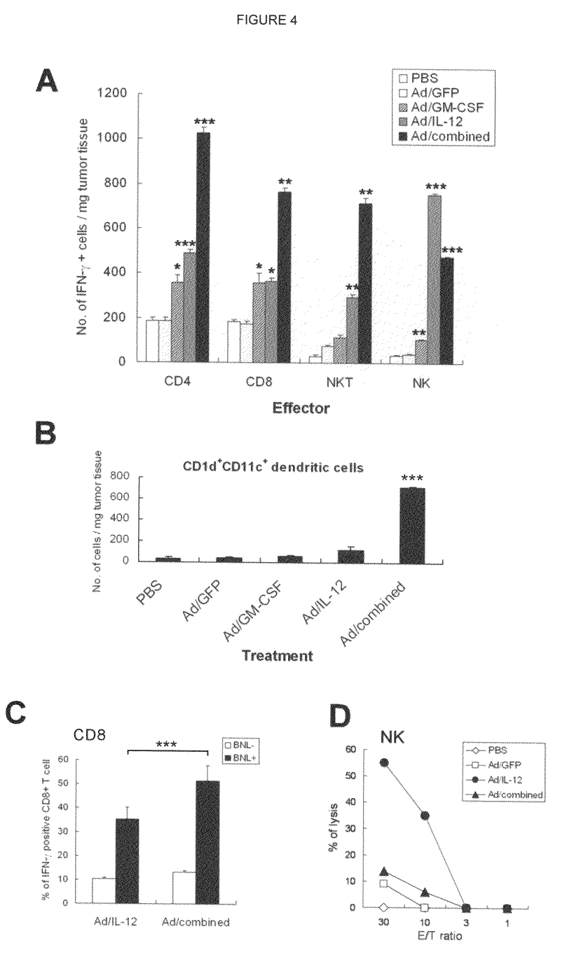

The markedly different results of subset depletion experiments between Ad/IL-12 and Ad/combined treatments prompted us to compare further the propensities of the effectors induced by these two treatments. Tumor infiltrating cells were isolated on day 4 after adenovirus injection and analyzed by flow cytometry. As illustrated in FIG. 4A, significantly higher levels of IFN-γ-secreting CD4+ T cells, CD8+ T cells, and NKT cells were detected in the tumor regions of the animals treated with Ad/combined than in the tumor regions of the animals treated with Ad/IL-12. In contrast, Ad/IL-12 treatment induced higher levels of NK infiltrates than Ad/combined treatment. As for Ad/GM-CSF treatment, it only induced moderately higher levels of CD4+ and CD8+ cells than Ad/GFP or PBS control treatment.

Activation of NKT cells requires the expression of CD1d molecule on the surface of antigen-presenting cells, e.g., dendritic cells (DCs). So, we measured the levels of CD1d+ DCs in tumor infiltrating cells. Data illustrated in FIG. 4B show that Ad/combined treatment induced markedly higher levels of CD1d+CD11c+ DCs than the other treatments. By activity assays, we further confirmed that Ad/combined treatment induced higher levels of tumor-specific CTLs than Ad/IL-12 treatment (FIG. 4C); whereas Ad/IL-12 treatment induced higher cytolytic NK activity than Ad/combined treatment (FIG. 4D). All these results are in good agreement with FIG. 4A's observations.

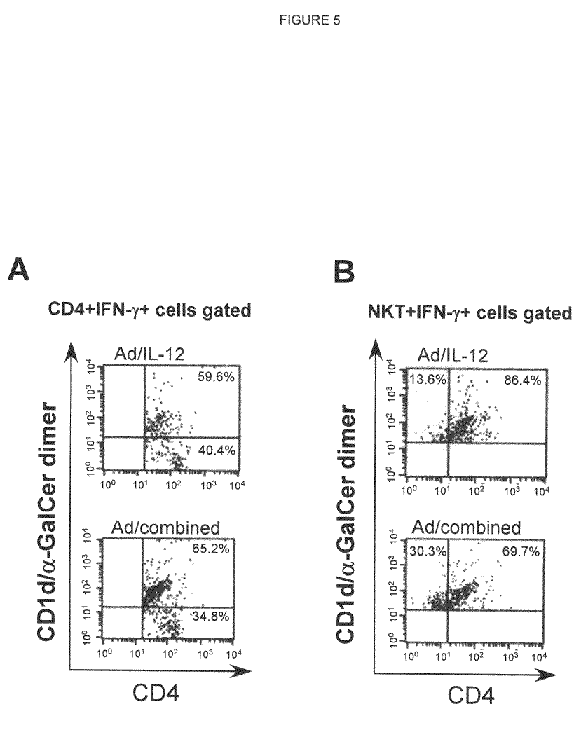

We further characterized the CD4+ population in tumor infiltrating lymphocytes by triply staining them with anti-IFN-γ, anti-CD4, and α-galactosylceramide (α-GalCer)-loaded CD1d:Ig DimerX I which stains NKT cells. It was found that in mice treated with Ad/IL-12, 59.6% of the CD4+ IFN-γ+ cells were NKT cells; whereas Ad/combined treatment further increased the NKT population to 65.2% (FIG. 5A). Moreover, we also found that in the activated NKT cells, the CD4− population, probably representing the CD4/CD8 double negative NKT cells, was significantly increased in Ad/combined treatment group compared to Ad/IL-12 treatment group (30.3% vs. 13.6%) (FIG. 5B). It was recently reported that the CD4/CD8 double negative NKT subset might exert higher antitumor activity than the CD4+ NKT subset (Crowe N Y et al. J Exp Med 2005; 202:1279-1288).

Collectively, these data indicate that GM-CSF and IL-12 combination therapy seems able to induce much higher levels of all types of effectors, except NK cells, than IL-12 monotherapy, which may explain why combination therapy has better antitumor effects than IL-12 monotherapy.

### Combination Therapy Induces Extraordinarily High Levels of Activated Macrophages in Tumor Regions

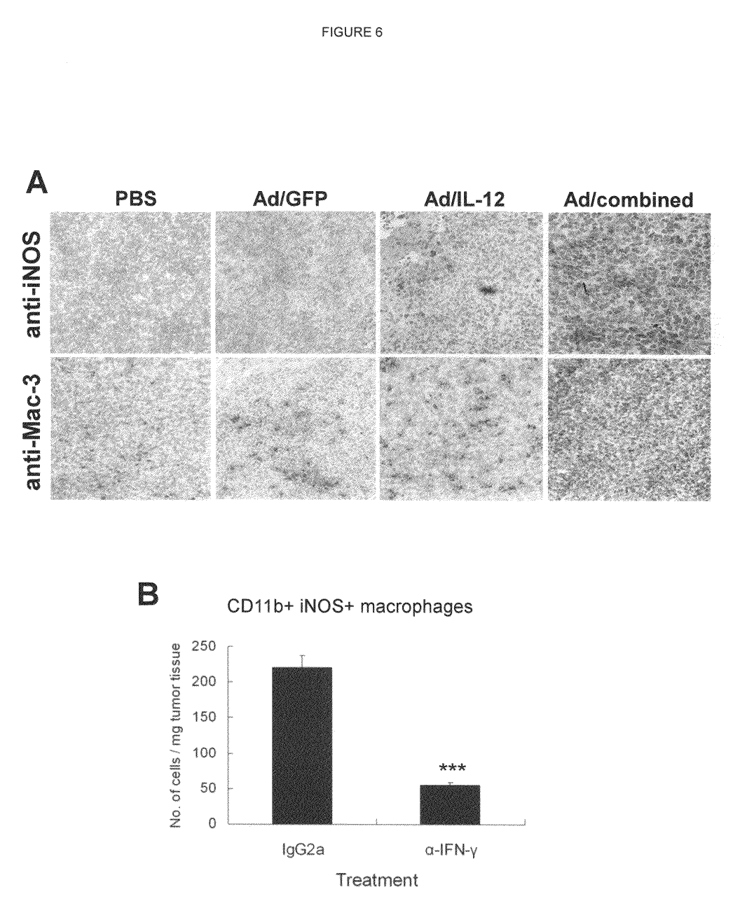

IFN-γ is also known to activate macrophages and induce them to produce NO by iNOS expression, which exhibits their tumoricidal activity (Adams DO and Hamilton TA. Annu Rev Immunol 1984; 2:283-318). So, we performed immunohistochemical staining with anti-Mac-3 or anti-iNOS antibody to detect any macrophages activated in tumor tissues of the animals treated with adenoviruses. Intense infiltration of activated macrophages was observed in tumor beds of the animals treated with Ad/combined, but much less in those with other treatments (FIG. 6A). The infiltration was in response to IFN-γ secretion, as macrophage infiltration was greatly reduced when animals were depleted of IFN-γ upon Ad/combined treatment. (FIG. 6B). Thus, tumoricidal macrophages were recruited and activated by the local high levels of IFN-γ.

### Example 2

### Treatment with Ad/ED or Ad/PEDF or Combined Treatment with Ad/ED, Ad/PEDF, Ad/IL-12 and Ad/GM-CSF (4-in-1 Therapy) Regresses Orthotopic Liver Tumors

Orthotopic liver tumors in male BALB/c mice or primary multifocal liver tumors in Wistar rats were induced in accordance with the procedure described above. Thereafter, a single injection of adenoviruses (2×10⁹ Ad/GFP, 2×10⁹ Ad/ED, 2×10⁹ Ad/PEDF, 1×10⁹ Ad/ED+1×10⁹ Ad/PEDF, or 0.5×10⁹ of each of Ad/GM-CSF, Ad/IL-12, Ad/ED, and Ad/PEDF), or 30 μl PBS was administered to the tumor bearing animals following the procedure described above.

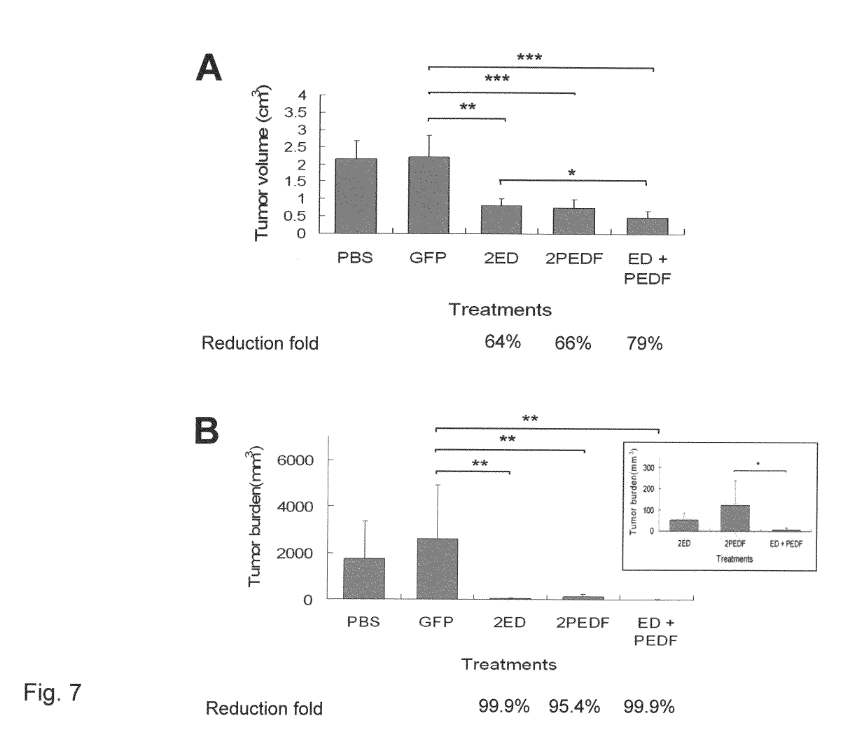

In mice model, animals that bear 7-day-old liver tumors, treatment with Ad/ED or Ad/PEDF alone reduced the tumor size for about 44% and 49%, respectively; whereas treatment with Ad/ED and Ad/PEDF at the same viral dose synergistically regressed the tumor size for about 79%, as compared with that of the control animals (FIG. 7A). In rat model, treatment with Ad/ED or Ad/PEDF alone, or with the combination of Ad/ED and Ad/PEDF, decreased the tumor size for about 99.9%, 95.4% and 99.9%, respectively (FIG. 7B).

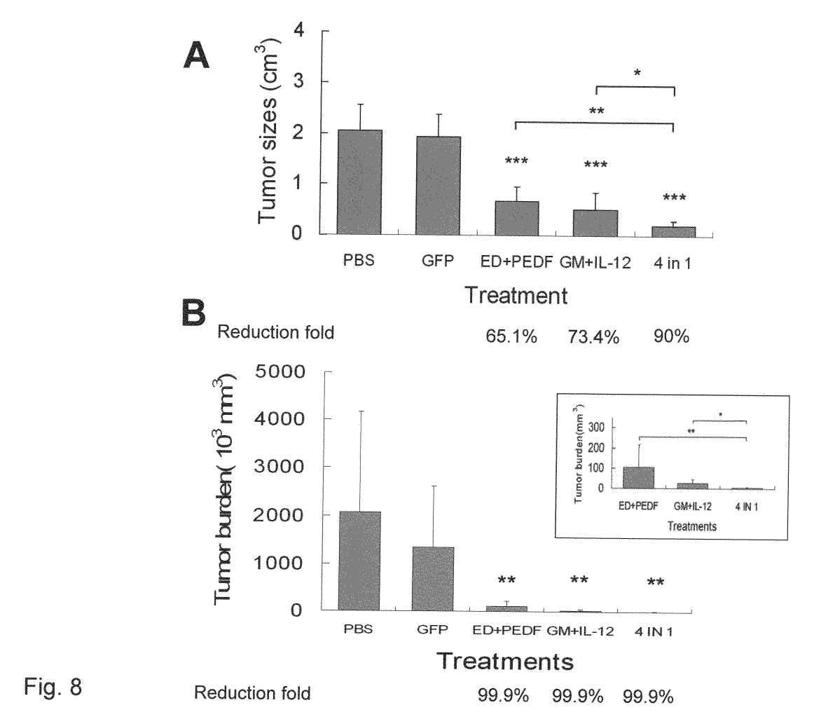

In mice with 14-day-old liver tumors, treatment with any of the followings, including the combination of Ad/ED and Ad/PEDF, the combination of Ad/IL-12 and Ad/GM-CSF, or the combination of Ad/ED, Ad/PEDF, Ad/IL-12 and Ad/GM-CSF (i.e., 4-in-1 therapy), reduced the tumor size for about 65.1%, 73.4% and 90%, respectively (FIG. 8A). Thus, combination of anti-angiogenic therapy with immunotherapy achieved a synergistic therapeutic effect, when compared with anti-angiogenic therapy alone or immunotherapy alone at the same viral dose. The same treatment was also effective to rats bearing multifocal liver tumors, as tumor size was successfully reduced for about 99.9%, 99.9% and 99.9%, respectively (FIG. 8B).

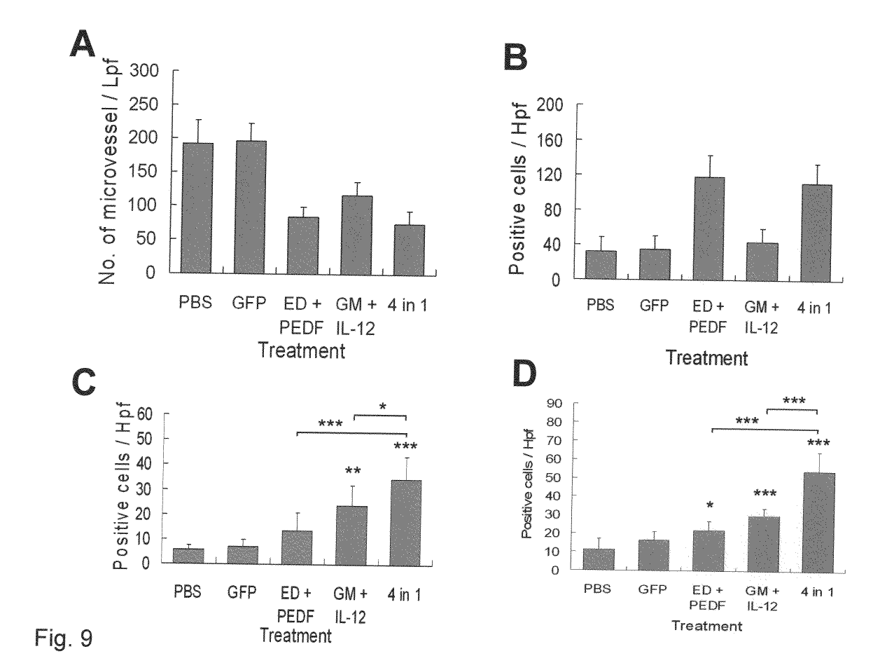

The combination of Ad/GM-CSF and Ad/IL-12, which had been proven to induce high levels of IFN-γ, was sufficient to reduce MVD although it was inferior to the combination of Ad/ED and Ad/PEDF; whereas the 4-in-1 therapy resulted in an even slightly lower tumor vascularization compared with the combination of Ad/ED and Ad/PEDF (FIG. 9A), suggesting that the 4-in-1 therapy synergistically reduced the MVD. The numbers of apoptotic cells in the tumor regions did not significantly increase by the combination of Ad/GM-CSF and Ad/IL-12, but did so by the combination of Ad/ED and Ad/PEDF and by the 4-in-1 therapy (FIG. 9B). Since the doses of Ad/ED and Ad/PEDF in the 4-in-1 treatment are only half of the combination of Ad/ED and Ad/PEDF, the similar levels of apoptotic cells in the 4-in-1 treatment may indicate that immunotherapy is able to enhance the induction of apoptosis of anti-angiogenesis therapy.

Immunohistochemical staining results indicate that significantly higher numbers of CD4 (+) (FIG. 9C) and CD8 (+)T cells (FIG. 9D) infiltrating into the tumor regions by the combined Ad/GM-CSF and Ad/IL-12 treatment, but not by the combined Ad/ED and Ad/PEDF treatment, compared to the control group. More importantly, the 4-in-1 treatment induced much higher levels of tumor infiltrating CD4 (+) (FIG. 9C) and CD8 (+) T cells (FIG. 9D) compared to the Ad/GM-CSF+Ad/IL-12 treatment. Hence, our results indicate that the combination of four factors provokes intense immune responses, which recruit high levels of effectors in the tumor regions, and they might contribute to the synergistic reduction of tumor volume.

In conclusion, this study demonstrates that combination therapy with any of the following combination, include but are not limited to GM-CSF and IL-12, ED and PDEF, and GM-CSF, IL-12, ED and PDEF, represents a promising strategy for treating orthotopic liver tumors. Remarkably, our preliminary results demonstrate that this strategy also exhibits significant antitumor effects on a woodchuck model with chronic hepatitis, which develops multifocal HCCs spontaneously. Thus, this combination mode may be considered as a potential therapeutic option to treat patients with widespread liver tumors.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for treating a cellular proliferative disorder in a subject, the method comprising administering to a subject in need thereof
an effective amount of a first polypeptide or a first nucleic acid encoding the first polypeptide, and
an effective amount of a second polypeptide or a second nucleic acid encoding the second polypeptide,
wherein the first polypeptide and the second polypeptide are selected from the group consisting of GM-CSF, IL-12, ED, and PEDF.

2. The method of claim 1, wherein the first nucleic acid and the second nucleic acid are adenoviral vectors.

3. The method of claim 1, wherein the cellular proliferative disorder is liver cancer.

4. The method of claim 2, wherein each polypeptide or nucleic acid is administered to a tissue or organ having the cellular proliferative disorder.

5. The method of claim 1, wherein first and the second polypeptides are GM-CSF and IL-12.

6. The method of claim 5, wherein the method further comprises administering to the subject a third polypeptide or a third nucleic acid encoding the third polypeptide, the third polypeptide being ED

7. The method of claim 6, wherein the method further comprises administering to the subject a forth polypeptide or a forth nucleic acid encoding the forth polypeptide, the forth polypeptide being PEDF.

8. The method of claim 7, wherein the cellular proliferative disorder is liver cancer.

9. The method of claim 8, wherein each polypeptide or nucleic acid is administered to a tissue or organ having liver cancer.

10. A pharmaceutical composition for treating a cellular proliferative disorder comprising a first polypeptide, a second polypeptide, and a pharmaceutically acceptable carrier, wherein the first polypeptide and the second polypeptide are selected from the group consisting of GM-CSF, IL-12, ED, and PEDF.

11. The composition of claim 10, wherein the first and the second polypeptides are GM-CSF and IL-12.

12. The composition of claim 10, wherein the cellular proliferative disorder is liver cancer.

13. The composition of claim 11, further comprising a third polypeptide, the third polypeptide being ED.

14. The composition of claim 12, further comprising a fourth polypeptide, the fourth polypeptide being PEDF.

15. The composition of claim 12, wherein the cellular proliferative disorder is liver cancer.

16. A pharmaceutical composition for treating a cellular proliferative disorder comprising a first nucleic acid encoding a first polypeptide, a second nucleic acid encoding a second polypeptide, and a pharmaceutically acceptable carrier, wherein the first polypeptide and the second polypeptide are selected from the group consisting of GM-CSF, IL-12, ED, and PEDF.

17. The composition of claim 15, wherein the first nucleic acid and the second nucleic acid are adenoviral vectors.

18. The composition of claim 16, wherein the first polypeptide encoded by the first nucleic acid is GM-CSF, and the second polypeptide encoded the second nucleic acid is IL-12.

19. The composition of claim 17, further comprising a third nucleic acid encoding a third polypeptide, the third polypeptide being ED.

20. The composition of claim 18, further comprising a fourth nucleic acid encoding a fourth polypeptide, the fourth polypeptide being PEDF.

21. The composition of claim 19, wherein the third nucleic acid and the fourth nucleic acid are adenoviral vectors.

22. The composition of claim 20, wherein the cellular proliferative disorder is liver cancer.
